(19) United States
(12) Patent Application Publication
(10) Pub. No.: US 2007/0117198 A1
(43) Pub. Date: May 24, 2007

(54) Industrial producing strain of the fungus Claviceps purpurea (Fr.) Tul.

(76) Inventors: Josef Valik, Opava (CZ); Tatana Kovarova, Ostrava-Poruba (CZ); Jiri Bohac, Opava (CZ)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 11/593,994

(22) Filed: Nov. 7, 2006

Related U.S. Application Data
(60) Provisional application No. 60/734,067, filed on Nov. 7, 2005.

(51) Int. Cl. C12N 1/16; C12N 15/74

(52) U.S. Cl. 435/254.1; 435/484

(57) ABSTRACT

Disclosed are strains of fungus Claviceps purpurea (Fr.) Tul., that produce a high content of total ergot peptide alkaloids and/or an amount of beta-ergokryptine greater than the amount of alpha-ergokryptine during parasitic growth on a cereal.

### CROSS-REFERENCE TO RELATED APPLICATIONS

The present application claims the benefit of the filing date of U.S. Provisional Application Ser. No. 60/734,067, filed Nov. 7, 2005, the disclosure of which is hereby incorporated by reference herein.

### FIELD OF THE INVENTION

The present invention relates to alkaloid producing strains of Claviceps purpurea. More specifically, it relates to strains of Claviceps purpurea (Fr.) Tul. that produce a high content of total ergot peptide alkaloids and an amount of beta-ergokryptine greater than the amount of alpha-ergokryptine during parasitic growth on a cereal.

### BACKGROUND OF THE INVENTION

Ergot peptide alkaloids, also known as ergopeptines, are natural products used for the manufacture of drugs. The category of ergopeptines includes a group of compounds known as ergokryptines that are useful in therapy. Ergokryptines include two isomeric forms: alpha-ergokryptine and beta-ergokryptine, both of which are used in medicinal formulations. The structure of alpha-ergokryptine was initially described by Stoll and Hofmann (Helv. Chim. Acta 26, 1570, 1943). The structure of beta-ergokryptine was initially described by Schlientz et al. (Experientia 23, 991, 1967).

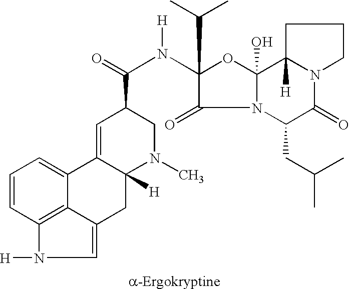

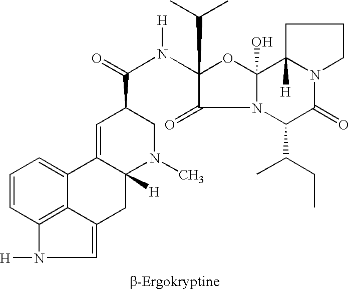

Ergokryptines are significant drug substances. In medicine, beta-ergokryptine and alpha-ergocryptine are used in dosage forms in combination with other active substances typically, frequently in a hydrogenated form as a component of codergocrine (mixture of mesilate salts of DH-ergocornine,—DH-ergokristine, DH-alpha-ergokryptine and DH-beta-ergokryptine see British Pharmacopoeia 2004, Vol. I, pp. 534-535). Alpha- and beta-dihydroergokryptine or 12′-hyrdoxy-2′-(1-methylethyl)-5′-alpha/beta-(2-methylpropyl)-9,10-dihydro-ergotaman-3′,6′,18-trione, are known compounds, derived from the hydrogenation of the double bond in position 9,10 of the natural alkaloid alpha- or beta-ergokryptine. Ergokryptines can also be used as a starting material for the manufacture of semi-synthetic alkaloid derivatives (for example, nicergoline, pergolide and cabergoline) . Drugs with these active substances, prepared from alkaloids isolated from sclerotia of the novel strain of Claviceps purpurea, have a broad spectrum of action and can be used as alpha-sympathicolytica, dopaminergic agents, antiserotonic agents, prolactininhibitors, antimigraine agents, antihypertensive agents, and in the treatment of peripheral metabolic and vascular disorders, such as, thromboangeitis obliterans, arteriosclerosis of cerebral vessels, senile cerebropathy and others known to the person skilled in the art.

Beta-ergokryptine and alpha-ergokryptine may be isolated from sclerotia produced by the fungus Claviceps purpurea (Fr.) Tul. when grown parasitically in field culture on rye or other cereals. A proprietary strain, kept in the Czech Collection of Microorganisms (CCM) under number CCM F-721, produces sclerotia having an average content of 0.56 wt % of total alkaloids to biomass dry weight during growth on rye. This strain is routinely used for the manufacture of beta-ergokryptine and alpha-ergokryptine (as described in CS 225 272 “Nový pr\dot{u}myslový kmen mikroorganismu Claviceps purpurea (Fr.) Tul. CCM F-721” (New industrial strain of microorganism Claviceps purpurea (Fr.) Tul., CCM F-721)—published only in Czech) . The ratio of alpha-ergokryptine to beta-ergokryptine produced by CCM F-721 is 1.15:1. Several other known strains of Claviceps purpurea, however, produce even higher amounts alpha-ergokryptine or produce the alpha form only. The preferred ratio of alpha-ergokryptine to beta-ergokryptine in commercial medicinal formulations is 2:1. Thus, a common practice in their preparation is to combine the fermentation products of an alpha-producing strain with those of a beta-producing strain. Use of a strain that produces relatively higher amounts of the beta form would be advantageous, therefore, because fewer strains would be needed to arrive at the preferred ratio of alpha- and beta-ergokryptines.

### SUMMARY OF THE INVENTION

Accordingly, in a first aspect of the present invention provides a strain of Claviceps purpurea (Fr.) Tul., or a mutant strain thereof, that produces an amount of beta-ergokryptine greater than the amount of alpha-ergokryptine during parasitic growth on a cereal, i.e., the weight ratio of beta-ergokryptine to alpha-ergokryptine is greater than 1:1. In a further aspect, the present invention provides a strain of Claviceps purpurea (Fr.) Tul., or a mutant strain thereof, that produces sclerotia containing a relatively high content of ergot peptide alkaloids relative to CCM F-721. In a yet further aspect, the present invention provides a strain of Claviceps purpurea (Fr.) Tul., (Ascension Number CCM 8360) or a mutant strain thereof, that produces sclerotia containing beta-ergokryptine and alpha-ergokryptine alkaloids during parasitic growth on a cereal. The present invention also provides a process for producing alpha-ergokryptine and beta-ergokryptine alkaloids which comprises culturing said strain of Claviceps purpurea. Methods of making mutants of CCM F-721 or CCM 8360 that possess these properties are also provided.

### DETAILED DESCRIPTION OF THE INVENTION

It has been surprisingly found that the ergot peptide alkaloid content is increased and the ratio of alpha-ergokryptine to beta-ergokryptine is changed in favour of beta-ergokryptine in a new strain of Claviceps purpurea (Accession Number CCM 8360) produced by a process of mutation and selection from strain CCM F-721.

The technical and scientific terms used herein have the meaning commonly understood by one of skill in the art to which the present invention relates, unless otherwise defined. Any suitable material or method known to those of skill in the art can be utilised in carrying out the present invention.

As used herein, “strain” refers to a population of cells, fungal tissues and filaments with the same phenotype and genotype characters based on genetic background.

As used herein, “sclerotium” refers to a step in the life cycle of parasitic Claviceps fungus. A sclerotium develops after the infection of an ovary by a Claviceps spore (see Tenberge, K. B. (1999): Biology and Life Strategy of the Ergot Fungi. In V. K\check{r}en and L. Cvak (Eds.): ERGOT. The Genus Claviceps. Harwood Acad. Publ., pp. 25-56.

As used herein, “Parasitic growth” refers to a part of life cycle of Claviceps purpurea that begins with infection of a cereal ovary by a spore, followed by sclerotium development and growth and finalized by formation of a mature sclerotium with maximal ergot alkaloids content (see Németh, É. (1999): Parasitic Production of Ergot Alkaloids. In V. K\check{r}en and L. Cvak (Eds.): ERGOT. The Genus Claviceps. Harwood Acad. Publ., pp. 303-320).

As used herein, “Saprophytic growth” refers to growth of Claviceps fungus in artificial conditions, in the context of the invention it is the growth of fungal filaments and the production of conidias (see Malinka, Z. (1999): Saprophytic cultivation of Claviceps. In V. K\check{r}en and L. Cvak (Eds.): ERGOT. The Genus Claviceps. Harwood Acad. Publ., pp. 321-371).

As used herein, “Conidia” refers to an asexual spore of various fungi. In the context of the present invention it is an asexual spore of Claviceps purpurea usable for infection of ovaries in cereal ears (see Németh, É. (1999): Parasitic Production of Ergot Alkaloids. In V. K\check{r}en and L. Cvak (Eds.): ERGOT. The Genus Claviceps. Harwood Acad. Publ., pp. 303-320 and Pa\check{z}outová, S. and Parbery, D. P. (1999): The Taxonomy and Phylogeny of Claviceps. In V. K\check{r}en and L. Cvak (Eds.): ERGOT. The Genus Claviceps. Harwood Acad. Publ., pp. 57-78).

For large scale field production of ergot peptide alkaloids, cereals or grasses of the family Poaceae, which flower with open inflorescence, may be used. Their use enables propagation of the ergot infection. Species of rye are mostly used, preferably cms (cytoplasmatic male sterility) rye or other varieties that do not have fertile pollen. (see Németh, É (1999): Parasitic Production of Ergot Alkaloids. In V. K\check{r}en and L. Cvak (Eds.): ERGOT. The Genus Claviceps. Harwood Acad. Publ., pp. 303-320 and Geiger, H. H. and Bausback, G. A. (1979) Untersuchungen ueber die Eignung pollensterilen Roggens zur parasitischen Mutterkornerzeugung. Z. Pflanzenzuechtg., 83, 163-175).

As used herein, “dry mass” refers to the resulting mass of sclerotias following desiccation for 2 hours at 100° C.

The present invention provides a strain of Claviceps purpurea (Fr.) Tul., that produces sclerotia containing beta-ergokryptine and alpha-ergokryptine alkaloids during parasitic growth on a cereal e.g. rye. This strain is believed to be non-naturally occurring.

The present invention also provides a strain of Claviceps purpurea (Fr.) Tul., or a mutant strain thereof, that produces sclerotia containing beta-ergokryptine and alpha-ergokryptine alkaloids during parasitic growth on a cereal. The content of beta-ergokryptine and alpha-ergokryptine alkaloids is high in comparison with the progenitor strain CCM F-721. The said strain produces more beta-ergokryptine than alpha-ergokryptine (i.e., in a weight ratio greater than 1).

The present invention further provides a strain of Claviceps purpurea (Fr.) Tul., (Accession Number CCM 8360) that produces sclerotia containing a relatively high content of ergot peptide alkaloids relative to CCM F-721, and beta-ergokryptine and alpha-ergokryptine alkaloids during parasitic growth on a cereal, in a weight ratio of greater than 1:1. Methods of making mutants of CCM F-721 or CCM 8360 that possess these properties are also provided.

A sample of the strain CCM 8360 has been deposited according to The Budapest Treaty in the International depositary authority Czech Collection of Microorganisms (CCM) at the Masaryk University, Tvrdého 14, Brno, Czech Republic. The strain produces sclerotia containing ergot peptide alkaloids, in particular beta-ergokryptine and alpha-ergokryptine, during parasitic growth on cereals. Typical examples of appropriate cereals are rye or triticale.

Typically, “high” refers to the amount of the ergot peptide alkaloids present in ergot sclerotias of a producing strain and indicates that the level in the producing strain is greater than in the parent strain (CCM F-721).

The ergot peptide alkaloid content in sclerotia produced by strain CCM 8360 is in the range 0.6 to 1.92 wt % of weight of total ergot peptide alkaloids of the biomass dry weight. Typically the content of total ergot peptide alkaloids ranges from 0.94 to 1.58 wt % with an average content of 1.38 wt % of total ergot peptide alkaloids in biomass dry weight. As used herein, “wt % ” refers to the weight of the dry biomass divided by the weight of the ergokryptine multiplied by 100. Unless otherwise stated % values relate to wt % values. The applicant has found that strain CCM F-721 produces sclerotia having an average content of 0.56 wt % total ergot peptide alkaloids to biomass dry weight, during growth on rye. This strain is routinely used for the manufacture of alpha-ergokryptine and beta-ergokryptine. The ratio of alpha-ergokryptine to beta-ergokryptine, is 1.15:1.

The content of ergot peptide alkaloids in sclerotia is in the range 37-43% alpha-ergokryptine, 47-58% beta-ergokryptine, and 5-10% ergocornine, with an average proportion 41% alpha-ergokryptine, 52% beta-ergokryptine, and 7% ergocornine.

The ratio of alpha-ergokryptine to beta-ergokryptine is in the range 0.63:1 to 0.91:1 with an average content of 0.79:1. This means that the CCM 8360 strain produces over 50 wt % beta-alkaloid when the ratio of alpha-ergokryptine to beta-ergokryptine is considered.

The content of beta-ergokryptine and alpha-ergokryptine is greater that 90 wt % of the total alkaloid content, typically beta-ergokryptine and alpha-ergokryptine are 90-96 wt % of the total alkaloid content.

A high content of ergot peptide alkaloids in sclerotia allows more efficient extraction and purification of the alkaloids. Extraction can be by a method of toluene extraction with further purification steps as described in Cvak, L. (1999): Industrial Production of Ergot Alkaloids. In V. K\check{r}en and L. Cvak (Eds.): ERGOT. The Genus Claviceps. Harwood Acad. Publ., pp. 373-410. Isolation of alkaloids from sclerotia with high alkaloids content can also be achieved using the method described in WO/2005082910. These alkaloids may be used for the production of drugs e.g. Secatoxin, DCCK, Derginal, D-Ergotox, Progeril, Geroplus, Zodalin, Permax, Nikotergoline, Sermion, Dopergin, Parlodel, Ergotop, Dostinex.

The strains of the present invention can be easily grown (as described in Example 1) and the resulting sclerotias may be harvested without any additional requirements when compared to the methods used for conventional ergot peptide alkaloid-producing strains. These methods can be scaled up.

The strains of the present invention allow existing extraction and isolation procedures as described in paragraph [0026] to be used in the manufacture of alpha- and beta-ergokryptine in sclerotias enables higher yields to be achieved during extraction.

Enhanced recovery is achieved due to the high content of alpha-ergokryptine and beta-ergokryptine in sclerotia when compared with the parent strain CCM F-721. For 1 kg of crude extract of alpha- and beta-ergokryptine and ergocornine to be produced 250 kg sclerotias of the strain CCM F-721 are required. If the strain of the present invention is used only 100-150 kg of sclerotias are required.

In field scale cultures, the yields vary from 700 kg/ha to 2200 kg/ha, with an average yield of 1000 kg/ha, when using the current agricultural methods of ergot cultivation.

### EXAMPLES

### Example 1

Growth Characteristics of Strain CCM 8360

Saprophytic Growth

During saprophytic growth in agar-thickened malt medium (35 g malt extract, 5 g peptone, 13 g agar, 1000 ml H₂O), strain Claviceps purpurea (Fr.) Tul. and strain CCM 8360 form colonies of sphacelus airy mycelium. The mycelium is initially white and smooth but after approximately 27 days of cultivation they become light beige and wrinkled. The mycelium consists of branched hyphae and conidia which are oval in shape and about 8 micrometers long and 5 micrometers wide.

Parasitic Growth

During parasitic growth on rye the fungus produces dark violet, pigmented sclerotia with an average weight of 33 mg. Individual sclerotia contain ergot alkaloids in an amount of 0.6 to 1.92 wt % weight of alkaloid in biomass dry weight. An average content of total alkaloids ranges from 0.94 to 1.58 wt % and is typically 1.38 wt % of which 47%-58 wt % of the total alkaloid is beta-ergokryptine.

Field Scale Growth

An aqueous suspension of conidia was prepared from the required amount of storable dry ergot inoculating preparation (see CS 276 530 “Zp\dot{u}sob výroby skladovatelných p\check{r}ípravk\dot{u}s vysokým obsahem \check{z}ivých spór vláknitých hub” (“Process for production of the storable preparations with the high content of living spores of filamentous fungi”)—published only in Czech) of the strain CCM 8360. Other types of inoculating preparations are disclosed in CS 243 009 “Zp\dot{u}sob fermenta\check{c}ní p\check{r}ípravy námelov\acute{e} o\check{c}kovací látky” (“Process of fermentation production of the ergot inoculating preparation”)—published only in Czech; in Éva Németh, É. (1999): Parasitic Production of Ergot Alkaloids. In V. K\check{r}en and L. Cvak (Eds.): ERGOT. The Genus Claviceps. Harwood Acad. Publ., pp. 303-320; and in Malinka, Z. (1999): Saprophytic cultivation of Claviceps. In V. K\check{r}en and L. Cvak (Eds.): ERGOT. The Genus Claviceps. Harwood Acad. Publ., pp. 321-371). Typically an inoculating machine is used as described by Németh, É. (1999) in Parasitic Production of Ergot Alkaloids. In V. K\check{r}en and L. Cvak (Eds.): ERGOT. The Genus Claviceps. Harwood Acad. Publ., pp. 303-320), to innoculate sterile rye (cms) cultivar Hyclaro ears in the heading phase of growth were inoculated with this conidia suspension so that 3.2×10¹¹ of germinative conidia were used per hectare.

At the time of rye ripening (ear drying), when sclerotia begin to release from their bed about 55 days post inoculation, the sclerotia were harvested using a combine harvester. A total harvest yielded 1094 kg/ha of dark violet sclerotia containing an average of 1.38 wt % of total alkaloids in dry biomass of sclerotias, 93 wt % of the total alkaloids were beta-ergokryptine and alpha-ergokryptine and 58% of the total alkaloid was beta-ergokryptine.

The resulting sclerotia were extracted as described in paragraph [0026], and the resulting extracts were used for the manufacture of peptidic ergot alkaloids or ergotoxine and/or for the manufacture of semi-synthetic alkaloids for use in the production of drugs in various dosage forms as described in Cvak, L.: Industrial Production of Ergot Alkaloids. In V. K\check{r}en and L. Cvak (Eds.): ERGOT. The Genus Claviceps. Harwood Acad. Publ. 1999, pp. 373-410.

### Example 2

Mutation and Selection (or Isolation) Process

A new industrial producer strain of the microorganism Claviceps purpurea (Fr.) Tul., CCM 8360, was bred from proprietary strain F-721. Sclerotium of this strain was transferred to saprophytic culture—a surface of sclerotium was sterilized and moved on agar-thickened malt medium. After 28 days at temperature 25° Celsius biomass of conidia covered the surface of the agar. The biomass was subjected to mutation using solution of 0.2M N-methyl-N-nitro-N-nitrosoguanidine for 8 hours. Mutated isolates were inoculated onto rye using a syringe, and grown sclerotia were analyzed for the qualitative and quantitative content of ergot alkaloids. Sclerotium with the highest content again was transferred to saprophytic culture that was subject to mutation using 0.2M ethyl methane sulphonate for 16 hours. Mutated isolates so obtained were inoculated onto rye using a syringe, and grown sclerotia were analyzed. Sclerotium with the highest content was passaged on rye in experiments in the small-plot scale (approx. 1 m²). Sclerotium of this strain was transferred to saprophytic culture that was exposed to Gamma radiation, with radiation dose being 3000 Gy. Irradiated isolates were inoculated onto rye using a syringe, and grown sclerotia were analyzed for the qualitative and quantitative content of ergot alkaloids. Sclerotium with the highest content was passaged on rye in pilot-scale experiments (approx. 1 ha) . Sclerotium with the highest content, harvested from these plots of land, was transferred to saprophytic culture and kept in the Czech Collection of Microorganisms (CCM) at the Masaryk University, Tvrdeho 14, Brno, Czech Republic, under identification number CCM 8360. The above mutation procedures connected with a positive selection resulted in an increase in the average content of ergot alkaloids in sclerotia by 146 wt % in strain CCM 8360, compared with initial strain CCM F-721. At the same time, the quality of the spectrum of individual alkaloids was improved by changing the ratio of alpha-ergokryptine to beta-ergokryptine from 1.15:1 to 0.79:1 in favour of desirable beta-ergokryptine.

Mutated isolates were then inoculated onto rye using a syringe. After growth of sclerotia had occurred, the sclerotia were analyzed for their qualitative and quantitative content of ergot alkaloids. Determination of the alkaloids content can be made by HPLC chromatography (column Hypersil ODS C18, 250×4.6 mm, mobile phase: 49.5% acetonitrile, 49.5% water, 1% triethylamine), or by liquid chromatography as described in European Pharmacopoeia 5.0, 2004, pp. 1347-1348. Sclerotium with the highest content of ergot alkaloids was transferred to saprophytic culture. The surface of the sclerotium was sterilized and moved on agar-thickened malt medium. After 28 days of cultivation at 25° C. biomass of conidia covered the surface of the agar. The conidia were mutated using a solution of 0.2M ethyl methane sulphonate in saline for 16 hours.

Mutated isolates were subsequently inoculated onto rye using a syringe and the resulting sclerotia were analyzed. The sclerotium with the highest ergot alkaloid content was passaged on rye in experiments on a small-plot scale (approx. 1 m²) . Sclerotium of this strain was transferred to saprophytic culture and exposed to gamma radiation with radiation dose being 3000 Gy. Irradiated isolates were inoculated onto rye using a syringe, then the resulting sclerotia were analyzed for the qualitative and quantitative content of ergot alkaloids as described in paragraph [0037].

The sclerotium with the highest content was passaged on rye in pilot-scale experiments (approx. 1 ha). Sclerotium with the highest content was harvested and transferred to saprophytic culture. The new strain is now deposited in the Czech Collection of Microorganisms (CCM) at the Masaryk University, Tvrdého 14, Brno, Czech Republic, under accession number CCM 8360.

The above mutation procedures when combined with the positive selection procedures resulted in an increase in the average content of ergot peptide alkaloids in sclerotia of 146 wt % for strain CCM 8360 when compared with progenitor strain CCM F-721. At the same time, the production of alpha- and beta-ergot peptide alkaloids was surprisingly improved by increasing the ratio of alpha-ergokryptine to beta-ergokryptine from 1.15:1 to 0.79:1 in favour of the more desirable beta-ergokryptine.

The same mutagenesis protocol may be applied to CCM F-721 or CCM 8360, followed by selection of a mutant strain that produces a content of ergot peptide alkaloids higher than CCM F-721, and/or more beta-ergokryptine than alpha-ergokryptine. Accordingly, this aspect of the invention is directed to a method for producing mutants of CCM F-721 and CCM 8360 and selecting or identifying mutants that produce a content of ergot peptide alkaloids higher than CCM F-721, and/or a higher amount of beta-ergokryptine than alpha-ergokryptine. The method entails mutagenizing CCM F-721 or CCM 8360, followed by analysis of the mutant to determine if it produces a content of ergot peptide alkaloids higher than CCM F-721, and/or a higher amount of beta-ergokryptine than alpha-ergokryptine. As illustrated in this example, mutagenesis is typically carried out by contacting or culturing CCM F-721 or CCM 8360 under suitable conditions with or in a medium containing a chemical mutagenizing agent and/or exposing the fungus to radiation.

In some embodiments, the method entails at least one round of mutagenesis of CCM F-721 or CCM 8360, wherein additional rounds of mutagenesis are conducted on sclerotium from the prior round that produce a content of ergot peptide alkaloids higher than CCM F-721, and/or a higher amount of beta-ergokryptine than alpha-ergokryptine. Thus, in some embodiments, the method entails mutagenizing CCM F-721 or CCM 8360, e.g., by transferring sclerotium of the strain to saprophytic culture; sterilizing a surface of the sclerotium was sterilized and transferring the sterilized portion to a suitable (e.g., agar-thickened malt) medium; culturing the sclerotium under suitable conditions of time and temperature (e.g., 28 days at temperature 250° Celsius); subjecting a biomass of conidia covered surface of the medium to a solution of a chemical mutagenizing agent (e.g., 0.2M N-methyl-N-nitro-N-nitrosoguanidine) for a suitable time (e.g., 8 hours) to produce a mutant of CCM F-721 or CCM 8360; disposing (e.g., inoculating) the mutant onto a cereal such-as rye; and analyzing sclerotia grown therefrom for the qualitative and quantitative content of ergot alkaloids; selecting sclerotium with the highest content of ergot peptide alkaloids, and transferring the selected sclerotium to saprophytic culture in the presence of a chemical mutagenizing agent (e.g., 0.2M ethyl methane sulphonate) for a suitable time (e.g., 16 hours), to produce a further mutant CCM F-721 or CCM 8360; disposing that mutant onto a cereal such as rye, and selecting among sclerotia grown therefrom for sclerotium with the highest ergot peptide alkaloid content; transferring the sclerotium to saprophytic culture and exposing the culture to radiation (e.g., Gamma radiation at a dose of 3000 Gy); disposing (e.g., inoculating) irradiated isolates onto a cereal such as rye and analyzing sclerotia grown therefrom for the qualitative and quantitative content of ergot peptide alkaloids.

### Example 3

Production of Nicergoline, Pergolide and Cabergoline from Alpha-Ergokryptine and Beta-Ergokryptine

The conversion of ergokryptine alkaloids to semi-synthetic alkaloid derivatives such as nicergoline, pergolide and cabergoline is well known in the art. First the ergokryptine alkaloid is converted into lysergic acid (Jacobs, W. A. and Craig, L. C.: The degradation of ergotinine with alkali. Lysergic acid. J. biol. Chem. 104, 547-551 (1934). The lysergic acid is then used to prepare the semi-synthetic ergot alkaloids according to the methods disclosed in EP 0003667 for pergolide, CZ 287047 for nicergoline and CZ 287176 for cabergoline. Cvak, L. (1999): Industrial Production of Ergot Alkaloids. In V. K\check{r}en and L. Cvak (Eds.): ERGOT. The Genus Claviceps. Harwood Acad. Publ., pp. 373-410 provides a review of the preparation of lysergic acid from ergokryptines and the preparation of semi-synthetic ergot alkaloids from lysergic acid.

All publications cited in the specification, both patent publications and non-patent publications are indicative of the level of skill of those skilled in the art to which this invention pertains. Any publication not already incorporated by reference herein is herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A strain of Claviceps purpurea (Fr.) Tul., that produces an amount of beta-ergokryptine greater than the amount of alpha-ergokryptine during parasitic growth on a cereal.

2. The strain of claim 1, wherein the cereal is a common cultivar of rye.

3. The strain of claim 1, wherein the common cultivar of rye is a cytoplasmatic male serility sterile rye cultivar.

4. The strain of claim 1, wherein the content of total alkaloids in the dry mass of sclerotia is 0.60 to 1.92 wt % of the dry mass.

5. The strain of claim 1, wherein the content of total alkaloids in dry mass has a content of 1.38 wt % .

6. The strain of claim 1, wherein the content of ergot peptide alkaloids in sclerotia is in the range 37-43 wt % alpha-ergokryptine, 47-58 wt % beta-ergokryptine, and 5-10 wt % ergocornine.

7. The strain of claim 1, wherein the content of ergot peptide alkaloids in sclerotia is 41 wt % alpha-ergokryptine, 52 wt % beta-ergokryptine, and 7 wt % ergocornine.

8. The strain of claims 1, wherein the ratio of beta-ergokryptine to alpha-ergokryptine is in the range 0.63:1 to 0.91:1.

9. The strain of claim 1, wherein the ratio of beta-ergokryptine to alpha-ergokryptine 0.79:1.

10. The strain of claim 1, wherein the content of beta-ergokryptine and alpha-ergokryptine in sclerotia is greater than 90% of the total ergot peptide alkaloid content.

11. The strain of claim 1, wherein the content of beta-ergokryptine and alpha-ergokryptine in sclerotia is 90%-96% of the total ergot peptide alkaloid content.

12. The strain of claims 1, wherein the yield of dry mass of sclerotia is in the range of 700 to 2200 kg/hectare.

13. The strain of claim 1, that produces sclerotia containing high content of ergot peptide alkaloids relative to CCM F-721.

14. The strain of claim 1, having an Accession Number CCM 8360.

15. A strain of Claviceps purpurea (Fr.) Tul., that has been deposited according to The Budapest Treaty in the International depositary authority Czech Collection of Microorganisms (CCM) at the Masaryk University, Tvrdého 14, Brno, Czech Republic under Accession Number CCM 8360.

16. A strain of Claviceps purpurea (Fr.) Tul., that produces sclerotia containing a high content of ergot peptide alkaloids relative to CCM F-721.

17. A strain of Claviceps purpurea derived from strain CCM F-721 having an average content of ergot peptide alkaloids that is higher than that present in CCM F-721.

18. The strain of Claviceps purpurea of claim 16, wherein the content of ergot peptide alkaloids is 0.6/0.56 wt % to 1.92/0.56 wt % higher than CCM F-721.

19. The strain of Claviceps purpurea of claim 18, wherein the content of ergot peptide alkaloids is 0.94/0.56 wt % to 1.58/0.56 wt % higher than CCM F-721.

20. A process for producing alpha-ergokryptine and beta-ergokryptine alkaloids which comprises culturing the strain of Claviceps purpurea of claim 1.

21. The process of claim 20 further comprising converting the ergokryptine alkaloids to semi-synthetic alkaloid derivatives such as alpha-dihydroergokryptine, beta-dihydrokryptine, nicergoline, pergolide and cabergoline.

22. The process of claim 20, further comprising admixing the ergokryptine alkaloids or semi-synthetic alkaloids with a pharmaceutical excipient.

23. A method for producing a mutant of CCM F-721 that produces a content of ergot peptide alkaloids higher than CCM F-721, and/or a higher amount of beta-ergokryptine than alpha-ergokryptine, comprising mutagenizing CCM F-721, thus producing a mutant of CCM F-721, and determining whether the mutant produces a content of ergot alkaloids higher than CCM F-721, and/or a higher amount of beta-ergokryptine than alpha-ergokryptine.

24. The method of claim 23, wherein said mutagenizing is performed by contacting CCM F-721 with a chemical mutagenizing agent.
